(19) United States
(12) Patent Application Publication
(10) Pub. No.: US 2024/0245917 A1
(43) Pub. Date: Jul. 25, 2024

(54) USE OF BLOOD FLOW IMAGES TO INFORM SCS WORKFLOW

(71) Applicant: Boston Scientific Neuromodulation Corporation, Valencia, CA (US)

(72) Inventors: Ismael Huertas Fernandez, Sevilla (ES); Que T. Doan, West Hills, CA (US)

(21) Appl. No.: 18/419,049

(22) Filed: Jan. 22, 2024

Related U.S. Application Data
(60) Provisional application No. 63/440,730, filed on Jan. 24, 2023.

(51) Int. Cl. A61N 1/36; G06T 7/00

(52) U.S. Cl. CPC A61N 1/36062; A61N 1/36135; G06T 7/0016; G06T 2207/10048; G06T 2207/30012

(57) ABSTRACT

A medical system may include a spinal cord stimulation (SCS) system, a thermographic imaging system, and an SCS suitability analyzer. The SCS system may be configured to deliver spinal cord neuromodulation including deliver a test SCS. The thermographic imaging system may be configured for taking thermal images of at least a portion of a patient. The SCS suitability analyzer may be configured to create thermographic imaging comparison data by comparing a thermographic imaging response to the test SCS to a thermographic imaging baseline before the test SCS is delivered, and analyze the thermographic imaging comparison data for an indicator of a significant perfusion response to the test SCS to determine whether the patient is a suitable candidate for SCS. A method may include capturing a thermographic imaging response to delivered SCS and performing, using the thermographic imaging response, a sweet spot determination for delivering the SCS.

### CLAIM OF PRIORITY

This application claims the benefit of U.S. Provisional Application No. 63/440,730 filed on Jan. 24, 2023, which is hereby incorporated by reference in its entirety.

### TECHNICAL FIELD

This document relates generally to medical systems, and more particularly, but not by way of limitation, to systems, devices, and methods for using blood flow images such as thermographic images with neuromodulation systems.

### BACKGROUND

Neural modulation has been proposed as a therapy for a number of conditions. Often, neural modulation and neural stimulation may be used interchangeably to describe excitatory stimulation that causes action potentials as well as inhibitory and other effects. Examples of neuromodulation include Spinal Cord Stimulation (SCS), Deep Brain Stimulation (DBS), Peripheral Nerve Stimulation (PNS), and Functional Electrical Stimulation (FES). SCS, by way of example and not limitation, has been used to treat chronic pain syndromes.

Although conventionally used as a pain therapy, SCS has also been suggested to improve perfusion for at least some patients who have ischemia such as chronic limb ischemia (CLI). It is desirable to provide improved systems and methods for determining if SCS is suitable for treating a patient's CLI.

Some neural targets may be complex structures with different types of nerve fibers. An example of such a complex structure is the neuronal elements in and around the spinal cord targeted by SCS. Furthermore, the number of available electrodes combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. It is desirable to provide improved systems and methods for finding a neuromodulation sweet spot.

### SUMMARY

A system may include a spinal cord stimulation (SCS) system configured to deliver spinal cord neuromodulation, and a blood flow imaging (BFI) system configured for generating images indicative of perfusion in at least a portion of a patient. The BFI system may include a camera-based system for detecting or estimating blood flow. The BFI system may include an ultrasound-based system for detecting or estimating blood flow. The system may be configured to use the BFI system to inform SCS system workflow. For example, the system may include an SCS suitability analyzer configured to create BFI comparison data by comparing a BFI response to the test SCS to a BFI baseline before the test SCS is delivered, and analyze the BFI comparison data for an indicator of a significant perfusion response to the test SCS to determine whether the patient is a suitable candidate for SCS. The BFI system may be used to capture a BFI response to delivered SCS, and use the BFI response to perform a sweet spot determination for delivering the SCS. Thermographic imaging systems are discussed herein a more specific example of a BFI system that are configured to generate images indicative of perfusion in at least a portion of the patient. The teachings provided herein discussed with respect to thermographic imaging systems may be applied to other BFI systems. For example, BFI may be substituted for the thermographic imaging referenced in the examples provided herein.

An example (e.g., Example 1) of a system may include a spinal cord stimulation (SCS) system, a thermographic imaging system, and an SCS suitability analyzer (which may be part of the SCS system, part of the imaging system or part of another device or system). The SCS system may be configured to deliver spinal cord neuromodulation including deliver a test SCS. The thermographic imaging system may be configured for taking thermal images of at least a portion of a patient. The SCS suitability analyzer may be configured to create thermographic imaging comparison data by comparing a thermographic imaging response to the test SCS to a thermographic imaging baseline before the test SCS is delivered. The SCS suitability analyzer may analyze the thermographic imaging comparison data for an indicator of a significant perfusion response to the test SCS to determine whether the patient is a suitable candidate for SCS.

In Example 2, the subject matter of Example 1 may optionally be configured such that the SCS system includes a programmer and a neuromodulator configured to deliver SCS including the test SCS, and the programmer of the SCS system includes the SCS suitability analyzer.

In Example 3, the subject matter of Example 1 may optionally be configured such that the thermographic imaging system includes an infrared camera operably connected to a display screen for displaying thermographic imaging, and the thermographic imaging system includes the SCS suitability analyzer.

In Example 4, the subject matter of Example 1 may optionally be configured such that the thermographic imaging system includes an infrared camera operably connected to a display screen for displaying thermographic imaging, and the SCS suitability analyzer includes a cloud-based analyzer implemented by one or more remote processing systems. The thermographic imaging system may be configured to capture and create digital image files for both the thermographic imaging baseline and the thermographic imaging response, and send the digital image files to the cloud-based analyzer.

In Example 5, the subject matter of Example 1 may optionally be configured such that the thermographic imaging system includes an infrared camera operably connected to a display screen for displaying thermographic imaging. The SCS suitability analyzer may include a cloud-based SCS suitability analyzer implemented by one or more remote processing systems. The SCS system may include a programmer and a neuromodulator configured to deliver SCS including the test SCS. The programmer may include a digital camera to capture thermographic imaging displayed on the display screen of the thermographic imaging system, and may be configured to use the digital camera create digital image files for both the thermographic imaging baseline and the thermographic imaging response and configured to send the digital image files to the cloud-based SCS suitability analyzer.

In Example 6, the subject matter of Example 1 may optionally be configured such that the thermographic imaging system includes an infrared camera operably connected to a display screen for displaying thermographic imaging. The system may further comprise a digital camera to capture thermographic imaging displayed on the display screen of the thermographic imaging system. The digital camera may be used to capture and create digital image files for both the thermographic imaging baseline and the thermographic imaging response when displayed on the display screen. The SCS suitability analyzer may be configured to use the digital image files to compare the thermographic imaging response to the test SCS.

In Example 7, the subject matter of Example 6 may optionally be configured to further include a personal device that includes the digital camera.

In Example 8, the subject matter of Example 7 may optionally be configured such that the personal device includes a phone or a tablet. The personal device may include a downloadable app. The SCS suitability analyzer may be implemented by personal device using the downloadable app to create and analyze the thermographic imaging comparison data from the digital image files. Alternatively, the SCS suitability analyzer may include a cloud-based SCS suitability analyzer implemented by one or more remote processing systems, and the personal device may use the downloadable app to send the digital image files to the cloud-based SCS suitability analyzer.

In Example 9, the subject matter of any one or more of Examples 1-8 may optionally be configured such that the SCS suitability analyzer is configured to create and analyze the thermographic imaging comparison data for a determined region of interest (ROI).

In Example 10, the subject matter of Example 9 may optionally be configured such that the SCS suitability analyzer is configured to determine the ROI by determining out-of-norm areas in the thermographic imaging baseline.

In Example 11, the subject matter of any one or more of Examples 9-10 may optionally be configured such that the SCS suitability analyzer is configured to receive user input identifying the determined ROI, and the user input includes a user selection of one or more of pre-defined regions or a user-defined ROI.

In Example 12, the subject matter of any one or more of Examples 1-11 may optionally be configured such that the SCS system is configured to receive real-time thermographic imaging feedback.

In Example 13, the subject matter of any one or more of Examples 1-12 may optionally be configured such that the SCS suitability analyzer is configured to store pre-SCS thermographic data corresponding the thermographic imaging baseline and SCS-response thermographic data corresponding to the thermographic imaging response, identify one or more pre-SCS statistical values for the pre-SCS thermographic data and one or more SCS-response statistical value(s) for the SCS-response thermographic data, determine one or more differences between the pre-SCS thermographic data and the SCS-response thermographic data, and compare the one or more differences to one or more threshold values to determine if the patient is suitable for SCS.

In Example 14, the subject matter of any one or more of Examples 1-12 may optionally be configured such that the SCS suitability analyzer is configured to store pre-SCS thermographic data corresponding the thermographic imaging baseline and SCS-response thermographic data corresponding to the thermographic imaging response, fit the pre-SCS thermographic data to a first distribution curve and fit the SCS-response thermographic data to a second distribution curve, identify a first set of one or more distribution parameter(s) for the first distribution curve and a second set of the one or more distribution parameters for the second distribution curve, determine one or more differences between corresponding one or more distribution parameters in the first set and the second set, and compare the one or more differences to one or more threshold values to determine if the patient is suitable for SCS.

In Example 15, the subject matter of Example 14 may optionally be configured such that the first distribution curve and the second distribution curves are gamma distributions, and the one or more distribution parameters include a shape parameter (k) and a scale parameter (Θ).

Example 16 includes subject matter (such as a method, means for performing acts, machine readable medium including instructions that when performed by a machine cause the machine to perform acts, or an apparatus to perform). The subject matter may include capturing a thermographic imaging baseline, using a thermal imaging system, before delivering a test spinal cord stimulation (SCS). The subject matter may include delivering the test SCS using an SCS system, and capturing a thermographic imaging response to the test SCS using the thermographic imaging system. The subject matter may include using an SCS suitability analyzer to create thermographic imaging comparison data by comparing the thermographic imaging response to the thermographic imaging baseline, analyze the thermographic imaging comparison data for a perfusion response, and determine if a patient is a suitable candidate for SCS based on the analyzed thermographic imaging comparison data.

In Example 17, the subject matter of Example 16 may optionally be configured such that the SCS system includes a programmer and a neuromodulator configured to deliver SCS including the test SCS, and the programmer of the SCS system includes the SCS suitability analyzer.

In Example 18, the subject matter of Example 16 may optionally be configured such that the thermographic imaging system includes an infrared camera operably connected to a display screen for displaying thermographic imaging, and the thermographic imaging system includes the SCS suitability analyzer.

In Example 19, the subject matter of Example 16 may optionally be configured such that the thermographic imaging system includes an infrared camera operably connected to a display screen for displaying thermographic imaging, and the SCS suitability analyzer includes a cloud-based analyzer implemented by one or more remote processing systems. The method may further include using the thermographic imaging system to capture and create digital image files for both the thermographic imaging baseline and the thermographic imaging response, and send the digital image files to the cloud-based analyzer.

In Example 20, the subject matter of Example 16 may optionally be configured such that the thermographic imaging system includes an infrared camera operably connected to a display screen for displaying thermographic imaging. The SCS suitability analyzer may include a cloud-based analyzer implemented by one or more remote processing systems. The method may further comprise using the thermographic imaging system to capture and create digital image files for both the thermographic imaging baseline and the thermographic imaging response, and send the digital image files to the cloud-based analyzer.

In Example 21, the subject matter of Example 16 may optionally be configured such that the thermographic imaging system includes an infrared camera operably connected to a display screen for displaying thermographic imaging. The system may further include a digital camera to capture thermographic imaging displayed on the display screen of the thermographic imaging system. The method may further include using the digital camera to capture and create digital image files for both the thermographic imaging baseline and the thermographic imaging response when displayed on the display screen, and using the digital image files to compare the thermographic imaging response to the test SCS.

In Example 22, the subject matter of Example 21 may optionally be configured such that a personal device includes the digital camera.

In Example 23, the subject matter of Example 22 may optionally be configured such that the personal device includes a phone or a tablet. The personal device may include a downloadable app. The method may include using the downloadable app to create and analyze the thermographic imaging comparison data from the digital image files, or using the downloadable app to send the digital image files to a cloud-based SCS suitability analyzer implemented by one or more remote processing systems.

In Example 24, the subject matter of any one or more of Examples 16-23 may optionally be configured such that the SCS suitability analyzer is used to create and analyze the thermographic imaging comparison data for a determined region of interest (ROI).

In Example 25, the subject matter of Example 24 may optionally be configured such that the ROI is automatically determined by automatically determining out-of-norm areas in the thermographic imaging baseline.

In Example 26, the subject matter of Example 24 may optionally be configured such that the ROI is determined using user input identifying the determined ROI. The user input may include a user selection of one or more of pre-defined regions or a user-defined ROI.

In Example 27, the subject matter of any one or more of Examples 16-26 may optionally be configured to further include programming neuromodulation using real-time thermographic imaging feedback.

In Example 28, the subject matter of any one or more of Examples 16-27 may optionally be configured to use the SCS suitability analyzer to store pre-SCS thermographic data corresponding the thermographic imaging baseline and SCS-response thermographic data corresponding to the thermographic imaging response, identify one or more pre-SCS statistical values for the pre-SCS thermographic data and one or more SCS-response statistical value(s) for the SCS-response thermographic data, determine one or more differences between the pre-SCS thermographic data and the SCS-response thermographic data, and compare the one or more differences to one or more threshold values to determine if the patient is suitable for SCS.

In Example 29, the subject matter of any one or more of Examples 16-28 may optionally be configured to use the SCS suitability analyzer to store pre-SCS thermographic data corresponding the thermographic imaging baseline and SCS-response thermographic data corresponding to the thermographic imaging response, fit the pre-SCS thermographic data to a first distribution curve and fit the SCS-response thermographic data to a second distribution curve, identify a first set of one or more distribution parameter(s) for the first distribution curve and a second set of the one or more distribution parameters for the second distribution curve, determine one or more differences between corresponding one or more distribution parameters in the first set and the second set, and compare the one or more differences to one or more threshold values to determine if the patient is suitable for SCS.

In Example 30, the subject matter of Example 29 may optionally be configured such that the first distribution curve and the second distribution curves are gamma distributions, and the one or more distribution parameters include a shape parameter (k) and a scale parameter (Θ).

Example 31 includes subject matter (such as a method, means for performing acts, machine readable medium including instructions that when performed by a machine cause the machine to perform acts, or an apparatus to perform). The subject matter may include capturing a thermographic imaging response to delivered spinal cord stimulation (SCS), and performing, using the thermographic imaging response, a sweet spot determination for delivering the SCS.

In Example 32, the subject matter of Example 31 may optionally be configured such that the sweet spot determination includes lead placement.

In Example 33, the subject matter of any one or more of Examples 31-32 may optionally be configured such that the sweet spot determination includes a determination of at least one spatial parameter for the stimulation.

In Example 34, the subject matter of any one or more of Examples 31-32 may optionally be configured such that the sweet spot determination includes a determination of at least one temporal pattern for the stimulation.

In Example 35, the subject matter of any one or more of Examples 31-34 may optionally be configured such that the capturing includes a real-time capturing of the thermographic imaging response and the sweet spot determination is performed using the real-time capturing of the thermographic imaging response.

A non-transitory machine-readable medium including instructions, which when executed by a machine, cause the machine to perform a method comprising creating thermographic imaging comparison data by comparing thermographic imaging response to thermographic imaging baseline. The thermographic imaging baseline corresponds to thermographic imaging before a test spinal cord stimulation (SCS). The thermographic imaging response corresponds to a thermographic imaging response to the test SCS. The method may include analyzing the thermographic imaging comparison data for a perfusion response, and determining if a patient is a suitable candidate for SCS based on the analyzed thermographic imaging comparison data. The instructions may further cause the machine to perform a method according to the subject matter in any one or more of Examples 17-30.

A non-transitory machine-readable medium including instructions, which when executed by a machine, cause the machine to perform a method comprising capturing a thermographic imaging response to delivered spinal cord stimulation (SCS), and performing, using the thermographic imaging response, a sweet spot determination for delivering the SCS. The instructions may further cause the machine to perform a method according to the subject matter in any one or more of Examples 32-35.

A system may include a spinal cord stimulation (SCS) system configured to deliver spinal cord neuromodulation, a thermographic imaging system configured for taking thermal images of at least a portion of a patient, and at least one processor configured to capture a thermographic imaging response to delivered spinal cord stimulation (SCS) and perform, using the thermographic imaging response, a sweet spot determination for delivering the SCS. The processor may be further configured to perform a method according to the subject matter in any one or more of Examples 32-35.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an embodiment of a neuromodulation system.

FIG. 2 illustrates an embodiment of a modulation device, such as may be implemented in the neuromodulation system of FIG. 1.

FIG. 3 illustrates an embodiment of a programming system such as a programming device, which may be implemented as the programming device in the neuromodulation system of FIG. 1.

FIG. 4 illustrates, by way of example, an implantable neuromodulation system and portions of an environment in which system may be used.

FIG. 5 illustrates, by way of example, an embodiment of a SCS system, which also may be referred to as a Spinal Cord Modulation (SCM) system.

FIG. 6 illustrates a system that includes both a SCS system and a thermographic imaging system.

FIG. 7 illustrates an example of a thermographic image that may be presented on the display of the thermographic imaging system of FIG. 6.

FIG. 8 illustrates a system that includes both a SCS system and a thermographic imaging system where a programmer within the SCS system is configured to perform processes to inform SCS workflow using information from the thermographic imaging system.

FIG. 9 illustrates a system that includes both a SCS system and a thermographic imaging system where the thermographic imaging system is configured to perform processes to inform SCS workflow using information from the thermographic imaging system.

FIG. 10 illustrates a system that includes both a SCS system and a thermographic imaging system where the SCS system is configured to communicate thermographic image information to a cloud-based system to inform SCS workflow.

FIG. 11 illustrates a system that includes both a SCS system and a thermographic imaging system where a programmer of the SCS system includes a digital camera for capturing a thermographic image presented on a display of the thermographic imaging system, and is further configured to communicate thermographic image information to a cloud-based system to inform SCS workflow.

FIG. 12 illustrates a system that includes both a SCS system and a thermographic imaging system where the thermographic imaging system is configured to communicate thermographic image information to a cloud-based system configured to inform SCS workflow.

FIG. 13 illustrates a system that includes a SCS system, a thermographic imaging system and a personal device such as a phone or tablet configured to inform SCS workflow using information from the thermographic imaging system.

FIG. 14 illustrates a system that includes a SCS system, a thermographic imaging system and a personal device configured to communicate thermographic image information to a cloud-based system to inform SCS workflow.

FIG. 15 illustrates, by way of example and not limitation, an embodiment of a method for determining whether a patient is suitable for treating CLI with SCS.

FIG. 16 illustrates, by way of example and not limitation, some techniques for determining a region of interest (ROI).

FIG. 17 illustrates, by way of example and not limitation, an example of an atlas-based user selection of a ROI.

FIG. 18 illustrates, by way of example and not limitation, an example of an operator-defined ROI.

FIG. 19 illustrates, by way of example and not limitation, a method for programming SCS using thermographic images as real-time feedback.

FIG. 20 illustrates, by way of example and not limitation, analysis of comparison data for SCS suitability.

FIG. 21 illustrates, by way of example and not limitation, a statical evaluation for analyzing thermographic data.

FIG. 22 illustrates, by way of example and not limitation, a statistical evaluation involving temperature distribution modeling.

FIG. 23 illustrates, by way of example and not limitation, a curve fitting process to a gamma curve.

FIG. 24 illustrates, by way of example and not limitation, some real-time sweet spot determinations for lead placement, for programming spatial parameters for the neuromodulation field, and for programming temporal parameters for the neuromodulation field.

FIG. 25 illustrates a system that includes both a SCS system 2500 and a blood flow imaging system, similar to the system illustrated in FIG. 6.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

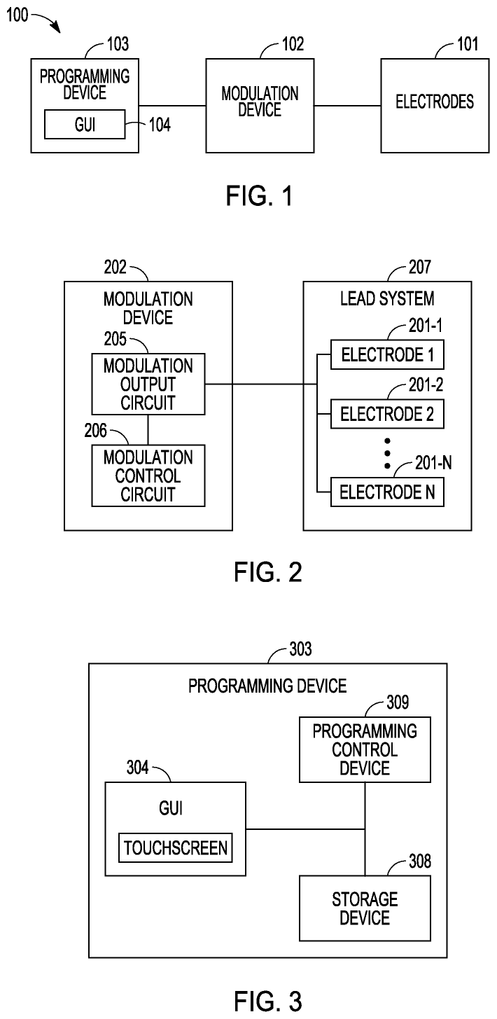

FIG. 1 illustrates, by way of example and not limitation, an embodiment of a neuromodulation system. The illustrated system 100 includes electrodes 101, a modulation device 102, and a programming system such as a programming device 103. The programming system may include multiple devices. The electrodes 101 are configured to be placed on or near one or more neural targets in a patient. The modulation device 102 is configured to be electrically connected to electrodes 101 and deliver neuromodulation energy, such as in the form of electrical pulses, to the one or more neural targets though electrodes 101. The delivery of the neuromodulation is controlled by using a plurality of modulation parameters. The modulation parameters may specify the electrical waveform (e.g. pulses or pulse patterns or other waveform shapes) and a selection of electrodes through which the electrical waveform is delivered. In various embodiments, at least some parameters of the plurality of modulation parameters are programmable by a user, such as a physician or other caregiver. The programming device 103 provides the user with accessibility to the user-programmable parameters. In various embodiments, the programming device 103 is configured to be communicatively coupled to modulation device via a wired or wireless link In various embodiments, the programming device 103 includes a graphical user interface (GUI) 104 that allows the user to set and/or adjust values of the user-programmable modulation parameters.

FIG. 2 illustrates an embodiment of a modulation device 202, such as may be implemented in the neuromodulation system 100 of FIG. 1. The illustrated embodiment of the modulation device 202 includes a modulation output circuit 205 and a modulation control circuit 206 . Those of ordinary skill in the art will understand that the neuromodulation system may include additional components such as sensing circuitry for patient monitoring and/or feedback control of the therapy, telemetry circuitry and power. The modulation output circuit 205 produces and delivers the neuromodulation. Neuromodulation pulses are provided herein as an example. However, the present subject matter is not limited to pulses, but may include other electrical waveforms (e.g. waveforms with different waveform shapes, and waveforms with various pulse patterns). The modulation control circuit 206 controls the delivery of the neuromodulation pulses using the plurality of modulation parameters. The lead system 207 includes one or more leads each configured to be electrically connected to modulation device 202 and a plurality of electrodes 201-1 to 201-N distributed in an electrode arrangement using the one or more leads. Each lead may have an electrode array consisting of two or more electrodes, which also may be referred to as contacts. Multiple leads may provide multiple electrode arrays to provide the electrode arrangement. Each electrode is a single electrically conductive contact providing for an electrical interface between modulation output circuit 205 and tissue of the patient, where N≥2. The neuromodulation pulses are each delivered from the modulation output circuit 205 through a set of electrodes selected from the electrodes 201-1 to 201-N. The number of leads and the number of electrodes on each lead may depend on, for example, the distribution of target(s) of the neuromodulation and the need for controlling the distribution of electric field at each target. In one embodiment, by way of example and not limitation, the lead system includes two leads each having eight electrodes. Some embodiments may use a lead system that includes a paddle lead.

The neuromodulation system may be configured to modulate spinal target tissue or other neural tissue. The configuration of electrodes used to deliver electrical pulses to the targeted tissue constitutes an electrode configuration, with the electrodes capable of being selectively programmed to act as anodes (positive), cathodes (negative), or left off (zero). In other words, an electrode configuration represents the polarity being positive, negative, or zero. An electrical waveform may be controlled or varied for delivery using electrode configuration(s). The electrical waveforms may be analog or digital signals. In some embodiments, the electrical waveform includes pulses. The pulses may be delivered in a regular, repeating pattern, or may be delivered using complex patterns of pulses that appear to be irregular. Other parameters that may be controlled or varied include the amplitude, pulse width, and rate (or frequency) of the electrical pulses. Each electrode configuration, along with the electrical pulse parameters, can be referred to as a “modulation parameter set.” Each set of modulation parameters, including fractionalized current distribution to the electrodes (as percentage cathodic current, percentage anodic current, or off), may be stored and combined into a modulation program that can then be used to modulate multiple regions within the patient.

The number of electrodes available combined with the ability to generate a variety of complex electrical waveforms (e.g. pulses), presents a huge selection of modulation parameter sets to the clinician or patient. For example, if the neuromodulation system to be programmed has sixteen electrodes, millions of modulation parameter sets may be available for programming into the neuromodulation system. Furthermore, for example SCS systems may have thirty-two electrodes which exponentially increases the number of modulation parameters sets available for programming To facilitate such selection, the clinician generally programs the modulation parameters sets through a computerized programming system to allow the optimum modulation parameters to be determined based on patient feedback or other means and to subsequently program the desired modulation parameter sets.

FIG. 3 illustrates an embodiment of a programming system such as a programming device 303, which may be implemented as the programming device 103 in the neuromodulation system of FIG. 1. The programming device 303 includes a storage device 308, a programming control circuit 309, and a graphical user interface (GUI) 304. The programming control circuit 309 generates the plurality of modulation parameters that control the delivery of the neuromodulation pulses according to the pattern of the neuromodulation pulses. In various embodiments, the GUI 304 includes any type of presentation device, such as interactive or non-interactive screens, and any type of user input devices that allow the user to program the modulation parameters, such as touchscreen, keyboard, keypad, touchpad, trackball, joystick, and mouse. The storage device 308 may store, among other things, modulation parameters to be programmed into the modulation device. The programming device 303 may transmit the plurality of modulation parameters to the modulation device. In some embodiments, the programming device 303 may transmit power to the modulation device. The programming control circuit 309 may generate the plurality of modulation parameters. In various embodiments, the programming control circuit 309 may check values of the plurality of modulation parameters against safety rules to limit these values within constraints of the safety rules.

In various embodiments, circuits of neuromodulation, including its various embodiments discussed in this document, may be implemented using a combination of hardware, software and firmware. For example, the circuit of GUI, modulation control circuit, and programming control circuit, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

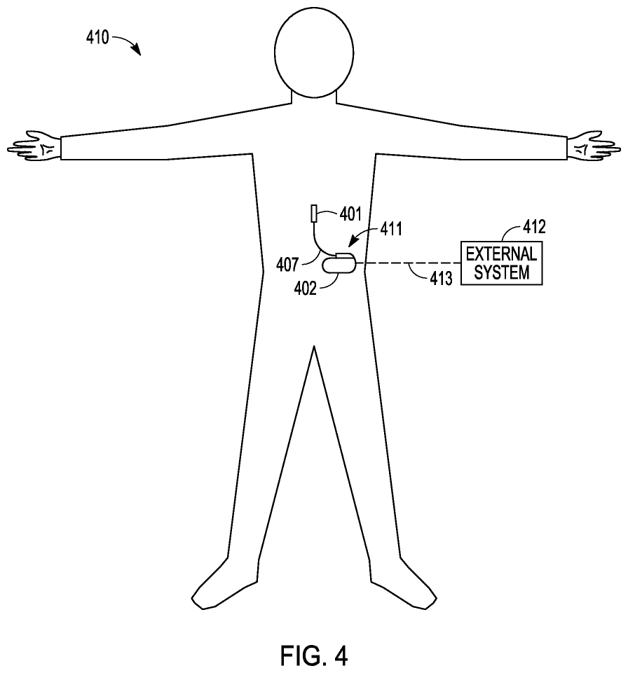

FIG. 4 illustrates, by way of example, an implantable neuromodulation system and portions of an environment in which system may be used. The system is illustrated for implantation near the spinal cord. However, neuromodulation system may be configured to modulate other neural targets. The system 410 includes an implantable system 411, an external system 412, and a telemetry link 413 providing for wireless communication between implantable system 411 and external system 412. The implantable system is illustrated as being implanted in the patient's body. The implantable system 411 includes an implantable modulation device (also referred to as an implantable pulse generator, or IPG) 402, a lead system 407, and electrodes 401. The lead system 407 includes one or more leads each configured to be electrically connected to the modulation device 402 and a plurality of electrodes 401 distributed in the one or more leads. In various embodiments, the external system 412 includes one or more external (non-implantable) devices each allowing a user (e.g. a clinician or other caregiver and/or the patient) to communicate with the implantable system 411. In some embodiments, the external system 412 includes a programming device intended for a clinician or other caregiver to initialize and adjust settings for the implantable system 411 and a remote control device intended for use by the patient. For example, the remote control device may allow the patient to turn a therapy on and off and/or adjust certain patient-programmable parameters of the plurality of modulation parameters. The external system 412 may include personal devices such as phones and tablets.

The neuromodulation lead(s) of the lead system 407 may be placed adjacent, i.e., resting near, or upon the dura, adjacent to the spinal cord area to be stimulated. For example, the neuromodulation lead(s) may be implanted along a longitudinal axis of the spinal cord of the patient. Due to the lack of space near the location where the neuromodulation lead(s) exit the spinal column, the implantable modulation device 402 may be implanted in a surgically-made pocket either in the abdomen or above the buttocks, or may be implanted in other locations of the patient's body. The lead extension(s) may be used to facilitate the implantation of the implantable modulation device 402 away from the exit point of the neuromodulation lead(s).

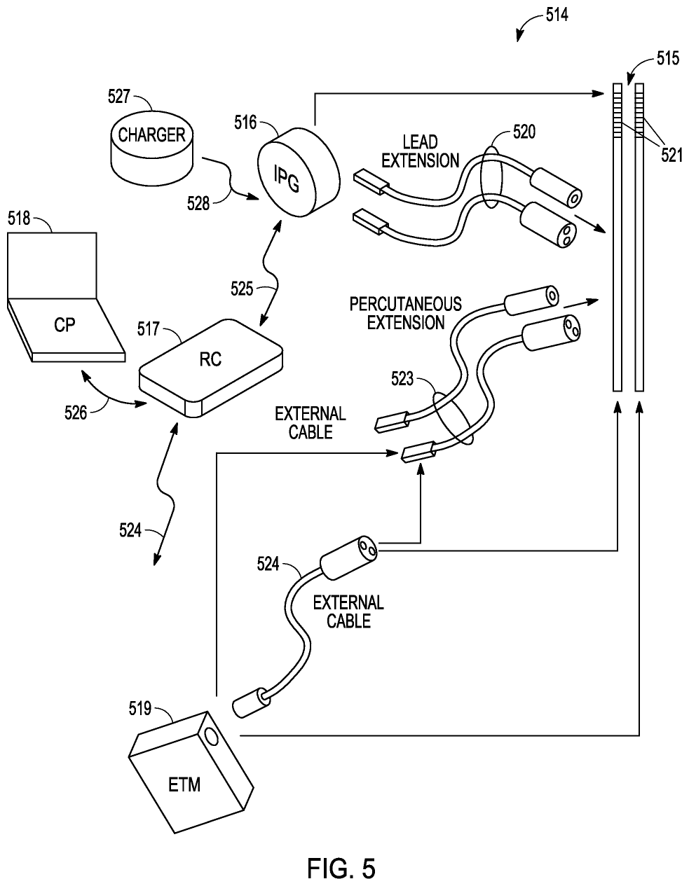

FIG. 5 illustrates, by way of example, an embodiment of a SCS system, which also may be referred to as a Spinal Cord Modulation (SCM) system. The SCS system 514 may generally include a plurality (illustrated as two) of implantable neuromodulation leads 515, an electrical waveform generator 516, an external remote controller (RC) 517, a clinician's programmer (CP) 518, and an external trial modulator (ETM) 519. IPGs are used herein as an example of the electrical waveform generator. However, it is expressly noted that the waveform generator may be configured to deliver regular, repeating patterns of pulses or in complex patterns that appear to be irregular patterns of pulses where pulses have differing amplitudes, pulse widths, pulse intervals, and bursts with differing number of pulses. It is also expressly noted that the waveform generator may be configured to deliver electrical waveforms other than pulses. The waveform generator 516 may be physically connected via one or more percutaneous lead extensions 520 to the neuromodulation leads 515, which carry a plurality of electrodes 521. As illustrated, the neuromodulation leads 515 may be percutaneous leads with the electrodes arranged in-line along the neuromodulation leads. Any suitable number of neuromodulation leads can be provided, including only one, as long as the number of electrodes is greater than two (including the waveform generator case function as a case electrode) to allow for lateral steering of the current. Alternatively, a surgical paddle lead can be used in place of one or more of the percutaneous leads. In some embodiments, the waveform generator 516 may include pulse generation circuitry that delivers electrical modulation energy in the form of a pulsed electrical waveform (i.e., a temporal series of electrical pulses) to the electrodes in accordance with a set of modulation parameters.

The ETM 519 may also be physically connected via the percutaneous lead extensions 522 and external cable 523 to the neuromodulation leads 515. The ETM 519 may have similar waveform generation circuitry as the waveform generator 516 to deliver electrical modulation energy to the electrodes accordance with a set of modulation parameters. The ETM 519 is a non-implantable device that is used on a trial basis after the neuromodulation leads 515 have been implanted and prior to implantation of the waveform generator 516, to test the responsiveness of the modulation that is to be provided. Functions described herein with respect to the waveform generator 516 can likewise be performed with respect to the ETM 519.

The RC 517 may be used to telemetrically control the ETM 519 via a bi-directional RF communications link 524. The RC 517 may be used to telemetrically control the waveform generator 516 via a bi-directional RF communications link 525. Such control allows the waveform generator 516 to be turned on or off and to be programmed with different modulation parameter sets. The waveform generator 516 may also be operated to modify the programmed modulation parameters to actively control the characteristics of the electrical modulation energy output by the waveform generator 516. A clinician may use the CP 518 to program modulation parameters into the waveform generator 516 and ETM 519 in the operating room and in follow-up sessions.

The CP 518 may indirectly communicate with the waveform generator 516 or ETM 519, through the RC 517, via an IR communications link 526 or other link The CP 518 may directly communicate with the waveform generator 516 or ETM 519 via an RF communications link or other link (not shown). The clinician detailed modulation parameters provided by the CP 518 may also be used to program the RC 517, so that the modulation parameters can be subsequently modified by operation of the RC 517 in a stand-alone mode (i.e., without the assistance of the CP 518). Various devices may function as the CP 518. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 518. Alternatively, such programming methodologies can be performed using firmware or hardware. In any event, the CP 628 may actively control the characteristics of the electrical modulation generated by the waveform generator 516 to allow the desired parameters to be determined based on patient feedback or other feedback and for subsequently programming the waveform generator 516 with the desired modulation parameters. To allow the user to perform these functions, the CP 518 may include a user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g. CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant waveform generator, implant waveform generator and lead(s), replace waveform generator, replace waveform generator and leads, replace or revise leads, explant, etc.), generate a pain map of the patient, define the configuration and orientation of the leads, initiate and control the electrical modulation energy output by the neuromodulation leads, and select and program the IPG with modulation parameters in both a surgical setting and a clinical setting.

An external charger 527 may be a portable device used to transcutaneously charge the waveform generator via a wireless link such as an inductive link 528. Once the waveform generator has been programmed, and its power source has been charged by the external charger or otherwise replenished, the waveform generator may function as programmed without the RC or CP being present.

SCS has conventionally been used as a pain therapy, SCS has also been suggested to improve perfusion for at least some patients who have ischemia such as chronic limb ischemia (CLI). Ischemia refers to a condition where blood flow is reduced to an organ or body part because of blockages or constriction of the blood vessels. Ischemia may cause a body part to have a shortage of oxygen needed for cellular metabolism. For example, lower extremity peripheral artery disease (PAD) refers to reduced blood flow that may be caused by plaque buildup in arteries of the leg. PAD may continue to develop until there are significant blockages in arteries (e.g., an advanced PAD stage which may be referred to as Critical Limb Ischemia (CLI)). CLI is a chronic condition that may result in amputation if pulsatile blood flow cannot be restored. Poor circulation may cause sores on the legs, and may prevent sores from healing on feet and toes. CLI often causes severe pain in the legs and feet.

The progression of CLI may be characterized using stages, such as the Fontaine stages. A first Fontaine stage may refer to asymptomatic conditions. A second Fontaine stage may refer to intermittent claudication (e.g., muscle pain when active and stopping when not active), and a third Fontaine stage may refer to ischemic pain at rest. A fourth Fontaine stage may refer to ulceration and/or gangrene.

Surgical procedures such as endovascular surgery may be attempted to restore adequate blood flow. However, surgery may not be effective or possible. SCS has been suggested for patients with CLI to provide both pain relief and improved blood flow as SCS decreases vascular resistance and relaxes smooth muscle (e.g., see Naoum J J, Arbid E J. Spinal cord stimulation for chronic limb ischemia. Methodist Debakey Cardiovasc J. 2013 April; 9(2):99-102. doi:10.14797/mdcj-9-2-99, PMID: 23805343; PMCID: PMC3693524; and Amann, W., Berg, P, Gersbach, P., Gamain, J., Raphael, J. H., Ubbink, D. Th. Spinal Cord Stimulation in the Treatment of Non-reconstructable Stable Critical Leg Ischaemia: Results of the European Peripheral Vascular Disease Outcome Study (SCS-EPOS). Eur J Vasc Endovasc Surg Vol 26, September 2003 280-286.)

SCS may improve some patient's condition, such as moving from a third or fourth Fontaine stage to a first or second Fontaine stage. Some CLI patients treated with SCS experience higher rates of limb survival. However, some CLI patients may not see significant improvement with SCS. For example, CLI may have progressed too far for SCS to significantly improve perfusion. SCS success in patients with CLI heavily depends on proper subject selection. Therefore, it is desirable to quantify a patient's condition to determine if SCS is suitable for the patient.

Transcutaneous oxygen pressure (TcpO₂) measurements have been suggested to evaluate trial SCS for treating CLI and were determined to have prognostic value in determining a positive microcirculatory response to SCS (see Amann, et al.). TcpO₂ measures local oxygen released from capillaries through the skin, and provides a measure of microcirculation, where values above 50 mmHg may be considered normal and values below 30 mmHg may be considered to indicate CLI. However, TcpO₂ or perfusion measurements are very discrete measurements as each measurement is a single body point, are time consuming (e.g., approximately 30 min for each measurement), and are scarce. An expensive machine that is not widely available in clinics and is only available in specialized clinics is used to measure TcpO₂.

MRI Angiography (e.g. MRA), a type of MRI used to evaluate narrowing or blockages of blood vessels, is also costly. The ankle-brachial index (ABI) test is time consuming. using a blood pressure cuff and ultrasound device for a clinician to listen for a pulse. The ABI checks for PAD by comparing blood pressure measured at the ankle with blood pressure measured at the arm. ABI is a measure of macrocirculation. An index (ankle blood pressure/arm blood pressure) between 0.9 and 1.3 may be considered normal, whereas a lower index may indicate PAD in the feet. For example, severe PAD may be indicated by an index between 0.00 to 0.40 and moderate PAD may be indicated by an index between 0.41 to 0.90.

The present subject provides, among other things, improved systems and methods for determining if SCS is suitable for treating a patient's CLI. For example, thermography may be used to quickly and economically provide quantification whether to proceed with SCS, which may both increase blood flow and improve pain relief. Unlike TcpO₂, thermographic technology is easily accessible, even available as a handheld infrared camera.

Thermography is a test that uses an infrared camera to detect heat patterns and blood flow in body tissues. The infrared camera functions as a heat sensor as it detects energy in the infrared portion of the energy spectrum. The detected heat may be displayed as a multicolor image. Different colors provide different temperature information. For example, green may be used to indicate a normal skin temperature, blue may indicate a cooler-than-normal skin temperature, red may indicate a warmer-than-normal skin temperature and white may indicated an even warmer skin temperature than the temperature represented by red. Thermography systems may allow different color palettes to be use, such as an iron palette (e.g., black (coldest), blue, read, orange yellow, white (hottest)), a black and white palette (black (coldest), white (hottest), and multiple levels of gray therebetween) and a rainbow palette (more color than the iron palette).

Each pixel of an infrared camera captures radiation from the targeted tissue. Some embodiments may transmit the captured wavelength information for each pixel for processing and/or may display a multicolor image to provide a visual representation of a combination of pixels. Skin with normal perfusion is expected to be within a normal temperature range, and skin with poor circulation is expected to have cooler than normal temperature.

Various embodiments may use an infrared camera (also referred to as a thermal imaging camera) to detect temperature changes as an indicator of the effectiveness of a trial SCS to treat CLI, and thus determine temperature metrics in patient tissue. For example, the infrared camera may be capable of accurately measuring body temperature and thus may be capable for being used to detect an increase in heat caused by an increase of flow of blood. Thermal imaging cameras may be hand-held devices, and thus thermographic imaging systems may be easily moved to the patient. The evaluated temperature metrics may be region-wise (an entire foot or a positive portion of the foot) or pixel-wise based on the smallest regions with detected radiation. The temperature metrics may indicate both a temperature before SCS and a temperature response to SCS. SCS suitability may be determined using metric cutoffs. Thermal imaging cameras may be hand-held devices, and thus thermographic imaging systems may be easily moved to the patient. Thermal imaging cameras may provide real-time information images an may be connected to other processing system (e.g., specialized software) for deeper analysis.

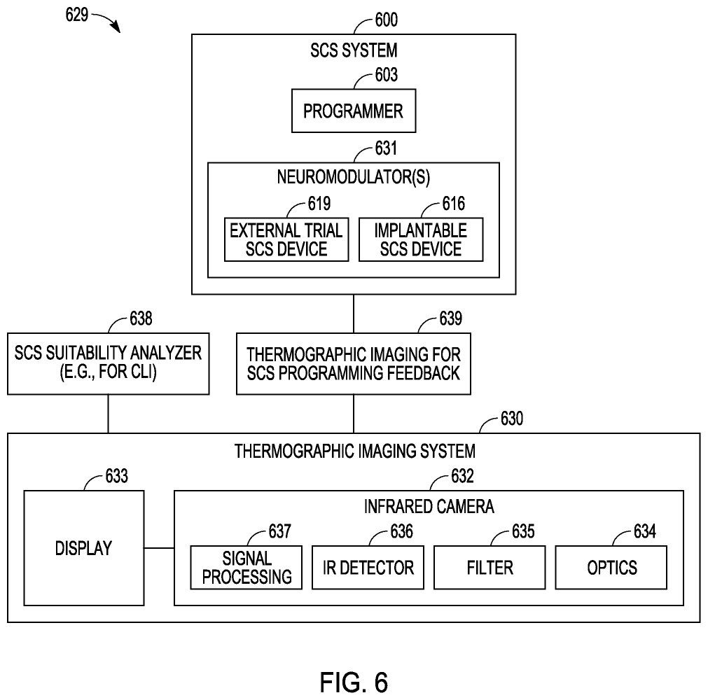

FIG. 6 illustrates a system 629 that includes both a SCS system 600 and a thermographic imaging system 630. The illustrated SCS system 600 may include a programmer 603 and neuromodulators 631, including an external trial SCS device 619 (e.g., ETM 519 in FIG. 5) used to test SCS and an implantable SCS device 616 (e.g., IPG 516 in FIG. 5). The thermographic imaging system 630 may generally include an infrared camera 632 and a display 633 used to display a thermographic image captured by the infrared camera 632. The infrared camera 632 may include components used to capture thermographic energy, including optics 634 to focus the infrared energy, a filter 635 to pass the wavelengths of interest, an infrared detector 636 configured to detect focused radiation from the targeted tissue and create individual pixels of image information that, in combination, form the overall thermographic image, and signal processing circuitry 637 which may be configured to process individual pixels and display the thermographic image. The illustrated system 629 may include an SCS suitability analyzer 638 which may, for example, be used to determine if SCS is suitable for a patient with CLI, and/or may include thermographic imaging feedback 639 (e.g., real-time images) such as may be used for finding sweet spots during programming or implantation procedures. The thermographic information may be received from the thermographic imaging system from the thermographic image on the display 633 and/or may be received as digital signals from the signal processing system 637. In some embodiments, a user may view the display 633 and use the thermographic image on the display to determine if the patient is suitable for SCS and/or provide feedback for finding sweet spots. In some embodiments the system may include processor(s) configured to analyze thermographic information (e.g., digital image information) transmitted in a communication signal.

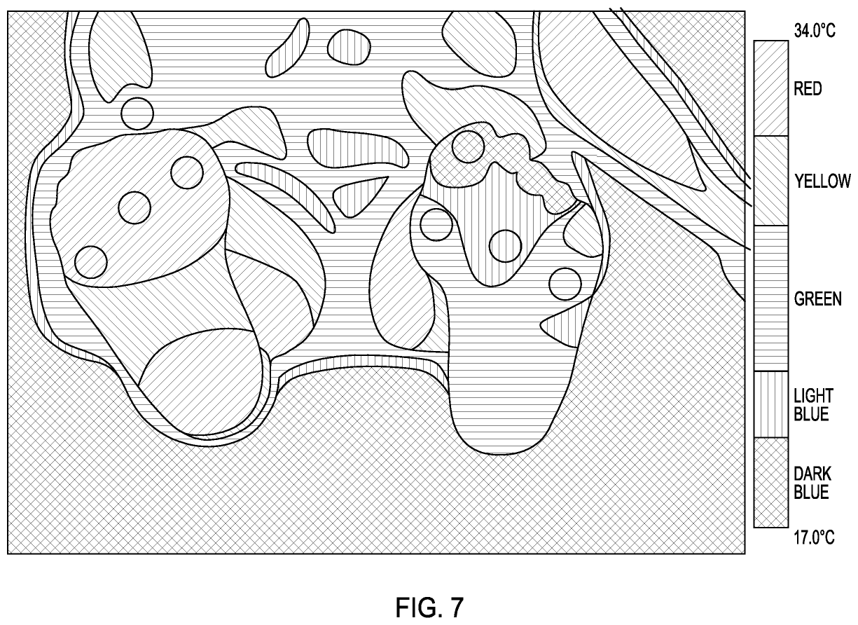

FIG. 7 illustrates an example of a thermographic image that may be presented on the display 633 of the thermographic imaging system 630 of FIG. 6. The thermographic image may typically be a multicolor image. For the purposes of this document and by way of example and not limitation, the image may be split into five regions, including a red region, yellow region, green region, light blue region and dark blue region, and these color regions are depicted in this drawing using different patterns. In the illustrated example, the left foot is generally colder than the right foot, and the four largest toes on the patient's left foot are significantly colder than other tissue indicating a region with significant ischemia. Some embodiments may analyze SCS suitability by determining whether the temperature of this ischemic region increases when SCS is delivered. Some embodiments may perform a sweet-spot analysis for the SCS by determining if temperature in a particular region increases when SCS is delivered.

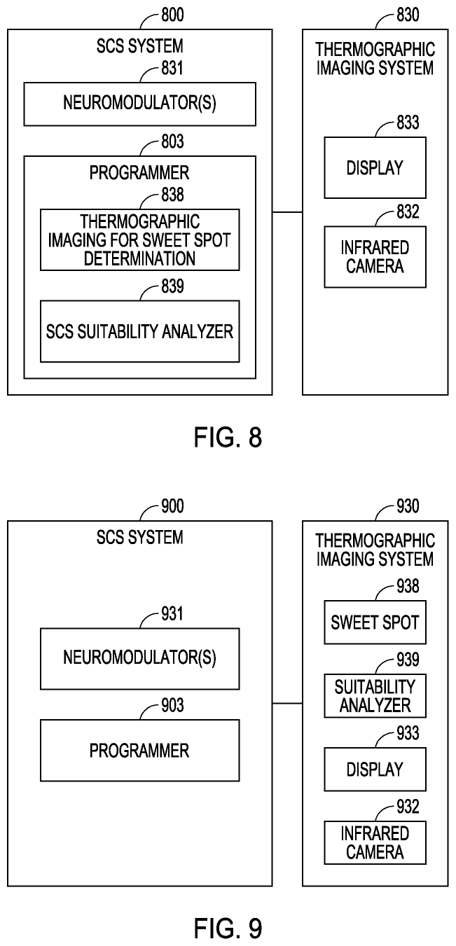

FIG. 8 illustrates a system that includes both a SCS system 800 and a thermographic imaging system 830 where a programmer 803 within the SCS system 800 is configured to perform processes to inform SCS workflow using information from the thermographic imaging system 830. The thermographic imaging system 830 may include an infrared camera 832 and a display 833, and the SCS system 800 may include one or more neuromodulators such as a trial neuromodulator and an implantable neuromodulator, and may further include a programmer 803. In some embodiments, the thermographic imaging system 830 is configured to communicate thermographic information to the programmer 803. The communication may include wireless and/or wired communication technology. The programmer 803 may be configured to process the thermographic information to provide real time, or near-real-time, sweet spot determination 838 during implantation procedures and/or programming. Additionally, or alternatively, the programmer 803 may be configured to process the thermographic information to analyze if SCS is suitable for a patient 839 (e.g., to treat CLI).

FIG. 9 illustrates a system that includes both a SCS system 900 and a thermographic imaging system 930 where the thermographic imaging system 930 is configured to perform processes to inform SCS workflow using information from the thermographic imaging system 930. The thermographic imaging system 930 may include an infrared camera 932 and a display 933, and the SCS system 900 may include one or more neuromodulators such as a trial neuromodulator and an implantable neuromodulator, and may further include a programmer 903. The thermographic imaging system 930 may be configured to process thermographic information to provide real time, or near-real-time, sweet spot determination 938 during implantation procedures and/or programming Additionally, or alternatively, the thermographic imaging system 930 may be configured to process the thermographic information to analyze if SCS is suitable for a patient 839, such as suitable to treat CLI. For example, the infrared camera 932 may be configured to process thermographic information for the sweet spot determination 938 and/or the suitability analysis 939.

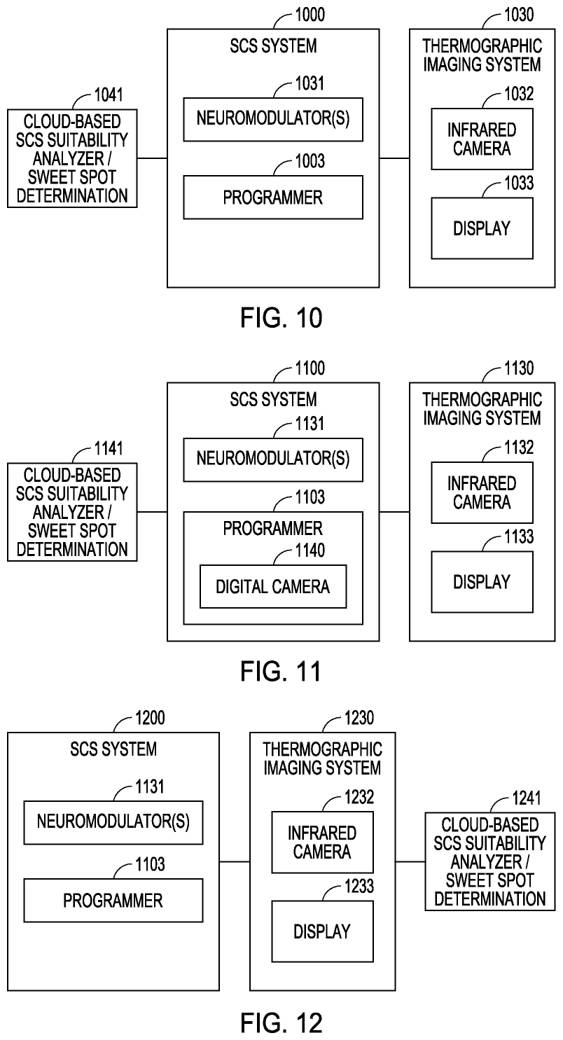

FIG. 10 illustrates a system that includes both a SCS system 1000 and a thermographic imaging system 1030 where the SCS system 1000 is configured to communicate thermographic image information to a cloud-based system 1041 to inform SCS workflow. The thermographic imaging system 1030 may include an infrared camera 1032 and a display 1033, and the SCS system 1000 may include one or more neuromodulators such as a trial neuromodulator and an implantable neuromodulator, and may further include a programmer 1003. In some embodiments, the thermographic imaging system 1030 is configured to communicate thermographic information to the programmer 1003, which may be configured to communicate thermographic information to the cloud-based system for providing processes to provide real time, or near-real-time, sweet spot determination during implantation procedures and/or programming, and/or may be configured to process the thermographic information to analyze if SCS is suitable for a patient, such as suitable to treat CLI. The cloud-based system 1041 may provide information back to the SCS system 1000 to inform the clinician or other operator to inform SCS workflow.

FIG. 11 illustrates a system that includes both a SCS system 1100 and a thermographic imaging system 1130 where a programmer 1140 of the SCS system 1100 includes a digital camera for capturing a thermographic image presented on a display of the thermographic imaging system, and is further configured to communicate thermographic image information to a cloud-based system 1141 to inform SCS workflow. The thermographic imaging system 1130 may include an infrared camera 1132 and a display 1133, and the SCS system 1100 may include one or more neuromodulators such as a trial neuromodulator and an implantable neuromodulator, and may further include a programmer 1103. The programmer 1103 may include a digital camera 1140, which may be used by the programmer 1103 to capture the thermographic image on the display 1133. The captured image may be communicated to the cloud-based system 1141. The cloud-based system 1141 may be configured to provide processes to provide real time, or near-real-time, sweet spot determination during implantation procedures and/or programming, and/or may be configured to process the thermographic information to analyze if SCS is suitable for a patient, such as suitable to treat CLI. The cloud-based system 1141 may provide information back to the SCS system 1100 to inform the clinician or other operator to inform SCS workflow.

FIG. 12 illustrates a system that includes both a SCS system 1200 and a thermographic imaging system 1230 where the thermographic imaging system 1230 is configured to communicate thermographic image information to a cloud-based system 1241 configured to inform SCS workflow. The thermographic imaging system 1230 may include an infrared camera 1232 and a display 1233, and the SCS system 1200 may include one or more neuromodulators such as a trial neuromodulator and an implantable neuromodulator, and may further include a programmer 1203. The thermographic imaging system 1230 may be configured to communicate thermographic image information to a cloud-based system 1241. The cloud-based system 1241 may be configured to provide processes to provide real time, or near-real-time, sweet spot determination during implantation procedures and/or programming, and/or may be configured to process the thermographic information to analyze if SCS is suitable for a patient, such as suitable to treat CLI. The cloud-based system 1241 may provide information back to the thermographic imaging system 1230 to inform the clinician or other operator to inform SCS workflow.

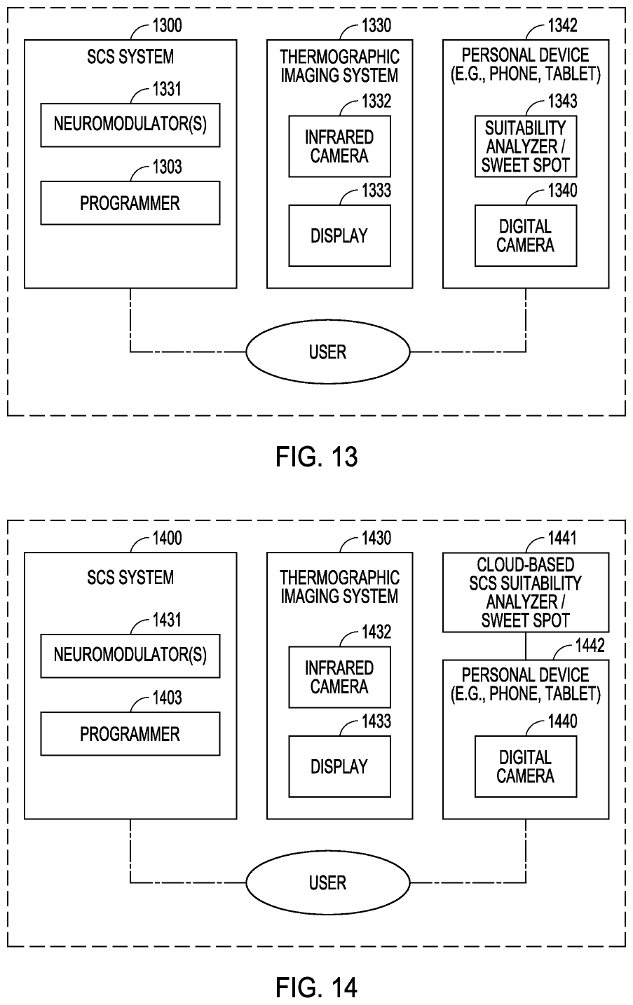

FIG. 13 illustrates a system that includes a SCS system 1300, a thermographic imaging system 1330 and a personal device such as a phone or tablet 1342 configured to inform SCS workflow using information from the thermographic imaging system 1330. The thermographic imaging system 1330 may include an infrared camera 1332 and a display 1333, and the SCS system 1300 may include one or more neuromodulators such as a trial neuromodulator and an implantable neuromodulator, and may further include a programmer 1303. The personal device 1342 may include a digital camera 1340 configured for use by the personal device to capture a thermographic image displayed on the display 1333 of the thermographic imaging system 1330. The personal device 1342 may be configured (e.g., a downloadable app 1343) to process the captured image to provide real time, or near-real-time, sweet spot determination during implantation procedures and/or programming, and/or may be configured to process the thermographic information to analyze if SCS is suitable for a patient, such as suitable to treat CLI. A user may use the processed information on the personal device to appropriately program the SCS system 1300.

FIG. 14 illustrates a system that includes a SCS system 1400, a thermographic imaging system 1430 and a personal device 1442 configured to communicate thermographic image information to a cloud-based system 1441 to inform SCS workflow. The thermographic imaging system 1430 may include an infrared camera 1432 and a display 1433, and the SCS system 1400 may include one or more neuromodulators such as a trial neuromodulator and an implantable neuromodulator, and may further include a programmer 1403. The personal device 1442 may include a digital camera 1440 configured for use by the personal device to capture a thermographic image displayed on the display 1433 of the thermographic imaging system 1430. The personal device 1442 may also be configured to communication thermographic imaging information to a cloud-based system 1441. The cloud-based system 1441 may be configured to provide processes to provide real time, or near-real-time, sweet spot determination during implantation procedures and/or programming, and/or may be configured to process the thermographic information to analyze if SCS is suitable for a patient, such as suitable to treat CLI. The cloud-based system 1441 may provide information back to the personal device 1442 to inform the clinician or other operator to inform SCS workflow.

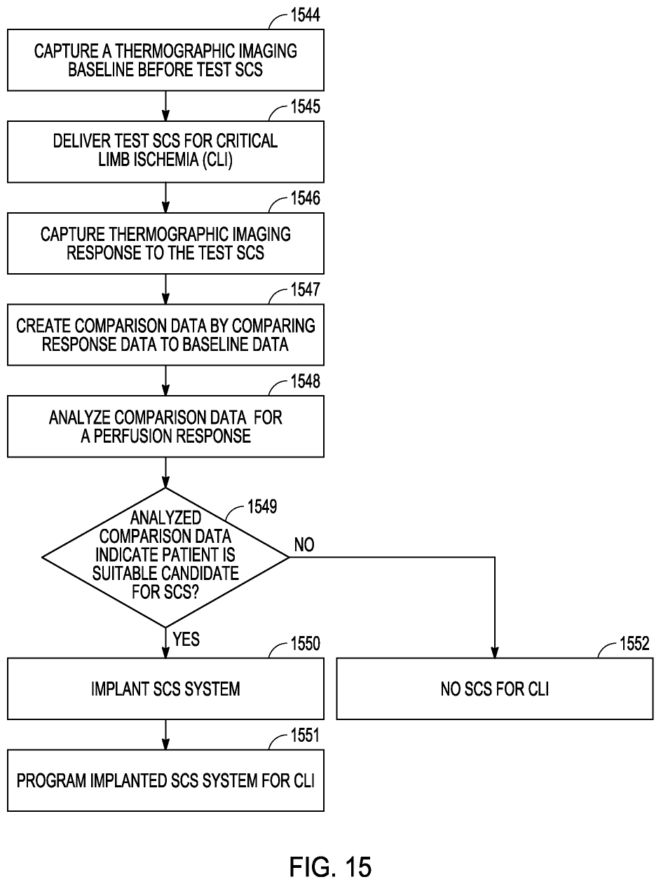

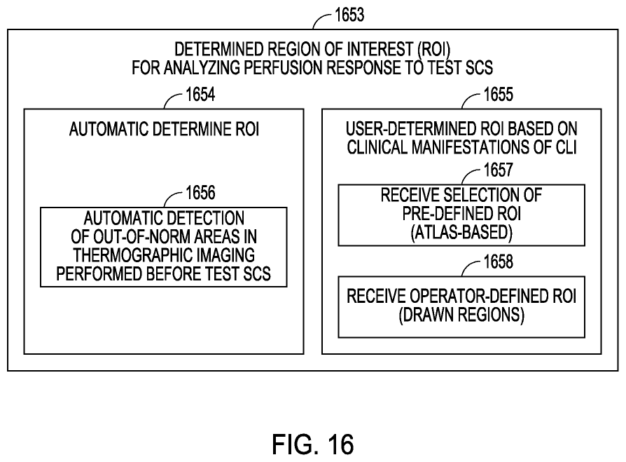

FIG. 15 illustrates, by way of example and not limitation, an embodiment of a method for determining whether a patient is suitable for treating CLI with SCS. The method may include capturing a thermographic imaging baseline before a test SCS is delivered 1544, and then delivering the test SCS for treating CLI 1545. At 1546, the method includes capturing a thermographic imaging response to the test SCS. The thermographic imaging response may be captured while the test SCS is being delivered. Some embodiments may end the test SCS before capturing the thermographic imaging response to the test SCS. At 1547, comparison data may be created by comparing thermographic imaging response data to the thermographic imaging baseline data. The comparison data may be analyzed for a perfusion response at 1548, and a determination is made whether the analyzed comparison data indicates whether the patient is a suitable candidate for SCS to treat CLI 1549. If, at 1549, it is determined that the patient is a suitable candidate, then the SCS system may be implanted at 1550 and programmed to deliver SCS to treat CLI at 1551. If, at 1549, it is determined that the patient is not a suitable candidate, then a decision may be made that the patient will not be treated with SCS to treat CLI.

Thermographic images may capture and/or analyze specific portions of the patient's body as a region of interest (ROI) for the purpose of determining a perfusion response, such as a perfusion response to test SCS for treating CLI. A ROI may be determined for a sweet spot analysis.

FIG. 16 illustrates, by way of example and not limitation, some techniques for determining the ROI. The ROI may be automatically determined 1654 or may be user-determined based on clinical manifestations of CLI 1655. Examples of clinical manifestations include: location of pain (e.g., ischemic rest pain); numbness; shiny, smooth or dry skin, and open sores, skin infections or ulcers. For example, the ROI may be automatically determined by an automatic detection of out-of-norm areas in thermographic imaging performed before the test SCS 1656. Normal temperature may be patient specific. For example, the patient's thermograph may have an overall temperature appearance where regions of temperature are within expected ranges of other regions of temperature. However, some regions may be significantly different (e.g., cooler) exceeding the expected range of other regions. Normal temperature may be based on data from larger patient populations, taking account of age, physical characteristics such as weight, body mass index, heath, comorbidities, and the like. A user may select an ROI from pre-defined ROIs, which may be presented in a pull-down list or regions on a programmer screen representing regions of the patient's tissue 1657. This may be referred to as an atlas-based user selection. A user or operator may define the ROI, such as by drawing or highlighting a region on a programmer screen representing a portion of the patient's tissue 1658. User selection may be based on patient-reported or clinically-observed manifestations of ischemia.

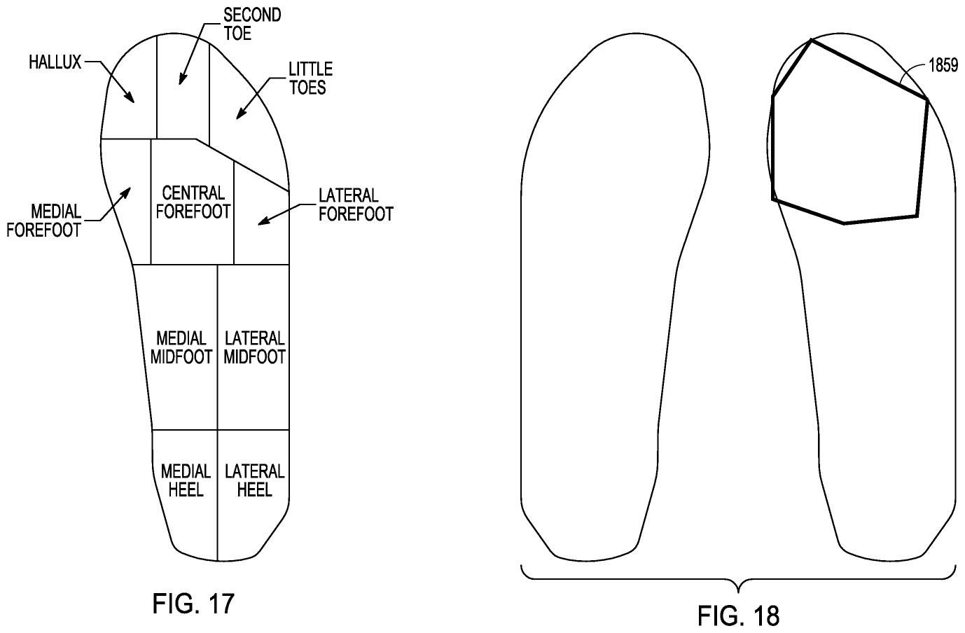

FIG. 17 illustrates, by way of example and not limitation, an example of an atlas-based user selection of a ROI. A GUI may display a region of the patient (e.g., foot), and may display predefined sub-regions of the foot. For example, predefined regions of the foot may include a hallux, second toe, little toes, medial forefoot, central forefoot, lateral forefoot, medial midfoot, lateral midfoot, medial heel and lateral heel. User selection of one or more regions may be based on patient-reported or clinically-observed manifestations of ischemia.

FIG. 18 illustrates, by way of example and not limitation, an example of an operator-defined ROI. A GUI may display a region of the patient (e.g., left and right feet), and may enable the operator to draw an outline 1859 to define an ROI. The ROI may be used to focus the infrared camera and/or may be used to control the portion of the thermal image to be compared or otherwise analyzed. The drawn outline 1859 may be based on patient-reported or clinically-observed manifestations of ischemia.

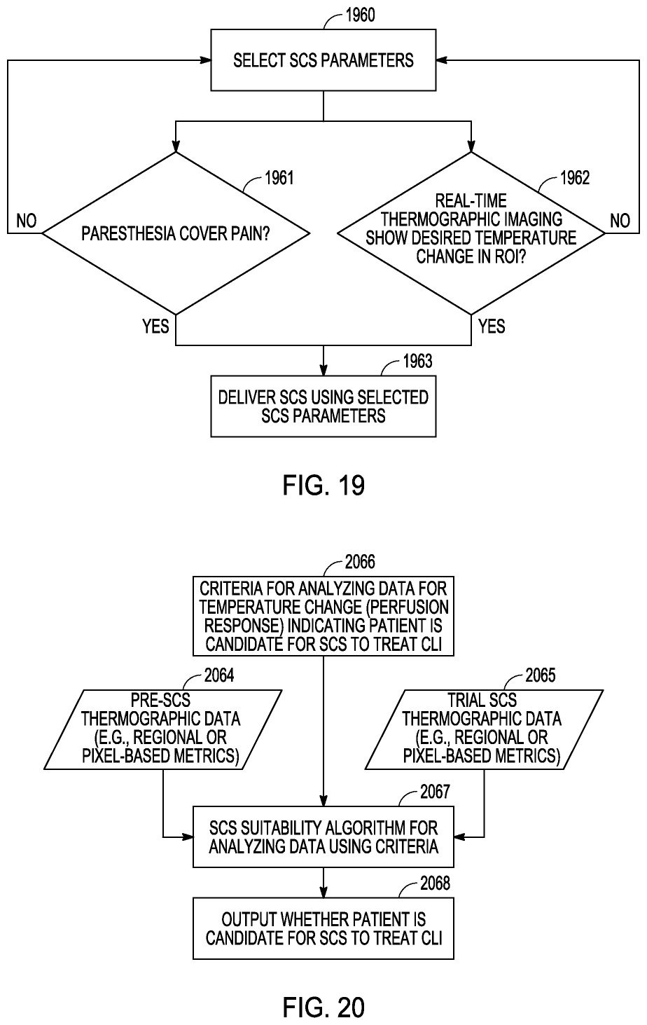

FIG. 19 illustrates, by way of example and not limitation, a method for programming SCS using thermographic images as real-time feedback. The SCS may be a test SCS or the SCS delivered by an implantable device. The method may include, at 1960, selecting SCS parameters. These SCS parameters may be parameters to be tested. At 1961 the method may include determining whether the SCS delivered using the selected SCS parameters covers the patient's pain. For example, paresthesia may be sensed where the patient was experiences pain. The patient's pain may be covered using sub-perception neuromodulation. If the patient's pain is not covered, the process may return to 1960 to select different SCS parameters. At 1962 the method may include determining whether real-time thermographic imaging is showing a desired temperature change in the ROI. If the real-time thermographic imaging is not showing a desired temperature change, then the process may return to 1960 to select different SCS parameters. Various embodiments may deliver SCS using the selected SCS parameters 1963 when the patient's pain is covered 1961 and/or when the real-time thermographic imaging shows a desired temperature change in the ROI 1962.

FIG. 20 illustrates, by way of example and not limitation, analysis of comparison data for SCS suitability. For example, both pre-SCS thermographic data 2064 and trial SCS thermographic data 2065 may be acquired. The data may be provided based on tissue regions or may be pixel-based. Criteria 2066 for analyzing data for temperature change may be acquired. The criteria may be specific for a perfusion response indicating that the patient is a candid for SCS to treat CLI or may be specific for another response to SCS.An SCS suitability algorithm 2067 may analyze the pre-SCS thermographic data 2064 and trial SCS thermographic data 2065 using the criteria 2066. The results of the analysis may be outputted 2068 to indicate whether the patient is suitable for SCS (e.g., whether the patient is a candidate for SCS to treat CLI).

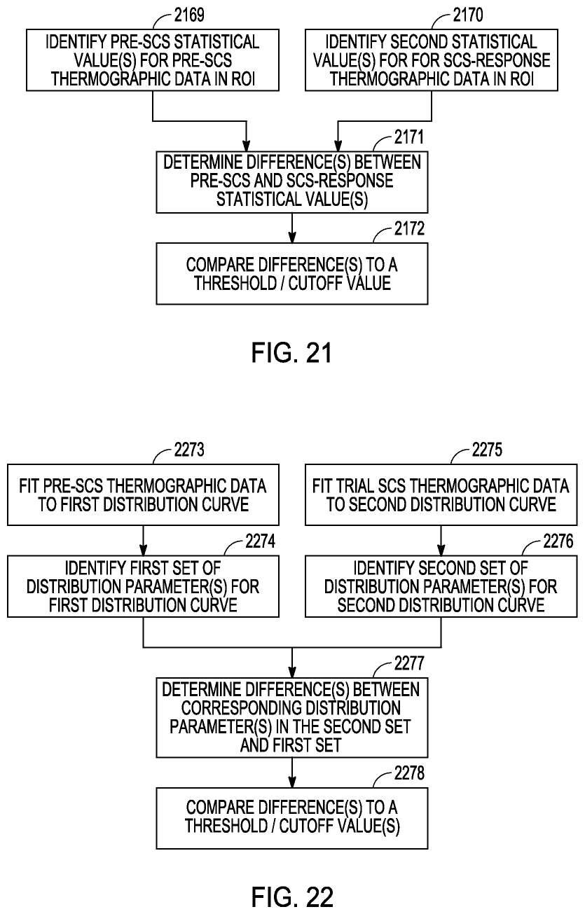

FIG. 21 illustrates, by way of example and not limitation, a statical evaluation for analyzing thermographic data. At 2169 pre-SCS statistical value(s) for pre-SCS thermographic data may be identified in the ROI, and at 2170 second statistical values for an SCS-response thermographic data may be identified in the ROI. The method may include determining a difference or differences between pre-SCS and SCS-response statistical value(s) 2171, and the difference(s) maybe compared to a threshold or cutoff value 2172. If the ROI is small enough or the number of evaluated data points is small, then a threshold change may be evaluated for relatively simple statistical values such as minimum value, maximum value, mean, median, and range. For example, if the ROI is focused on a region of unusually low temperature indicating poor perfusion, then the analysis may simply be determining whether the lowest temperature in the pre-SCS thermographic data increases by a threshold or determining if the average temperature increases by a threshold. By way of example and not limitation, the system may be designed to determine the lowest X % of the values, and analyze whether those values show a temperature increase (e.g., average increase) above a threshold. Other statistical techniques may be implemented.

FIG. 22 illustrates, by way of example and not limitation, a statistical evaluation involving temperature distribution modeling. For example, pre-SCS thermographic data may be fit to a first distribution curve at 2273 and a first set of distribution parameter(s) may be identified for the first distribution curve at 2274. Trial or test SCS thermographic data may be fit to a second distribution curve at 2275 and a second set of distribution parameter(s) may be identified for the second distribution curve at 2276. The difference(s) between corresponding distribution parameter(s) in the second and first sets may be determined at 2277. These differences reflect a change in skin temperature caused by a change in perfusion from the test SCS. These difference(s) may be compared to a threshold value or cutoff value to determine if SCS is suitable to treat the patient's CLI 2278.

By way of example and not limitation, a temperature distribution for a body region (e.g., injured foot) may be fit to a normal or gamma distribution curve. The distribution curves may include probability density function (pdf) or cumulative distribution function (cdf) curves. Probability density function identify the probability that a random variable takes on a certain value, and cumulative distribution functions identify the probability that a random variable takes on a value less than or equal to a certain value). That is, pdf is the derivative of cdf. By way of example, distribution parameters that may be analyzed if the data is fitted to a normal curve may include the mean and standard distribution, and distribution parameters that may be analyzed if the data is fitted to a gamma curve may include shape, scale and threshold (smallest value in the gamma distribution). The determined change in such distribution parameters may be compared to a cutoff value to determine SCS eligibility.

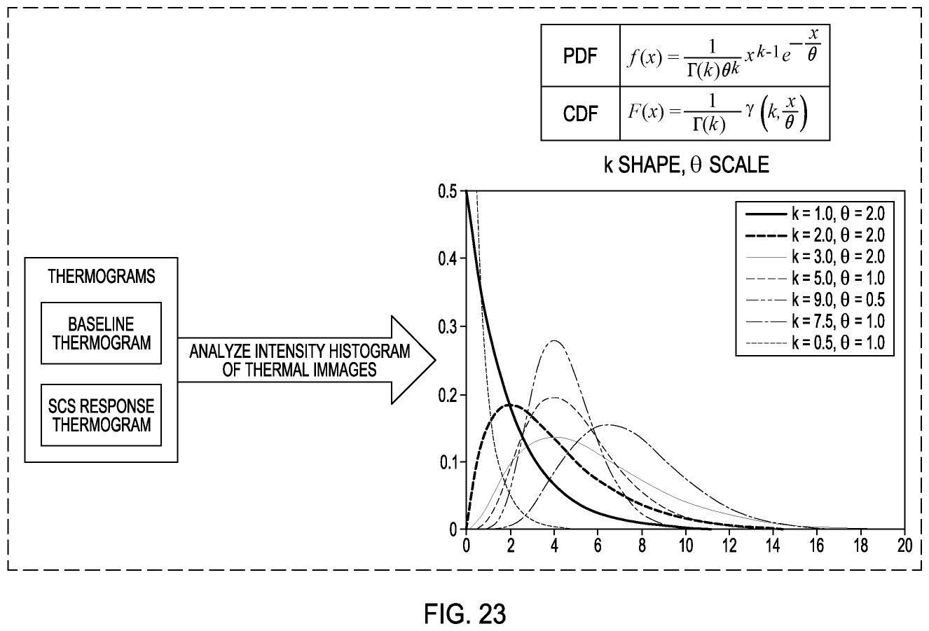

FIG. 23 illustrates, by way of example and not limitation, a curve fitting process to a gamma curve. Baseline and SCS-response thermographic images, also referred to as a thermograms, are analyzed. More particularly, an intensity histogram of each of the thermal images are analyzed to fit into one of the gamma curves. A shape parameter (k) and a scale parameter (Θ) may be determined for each of the thermal images based on the curve fittings. The change or difference in these parameters may be compared to a threshold to determine if SCS will likely be effective in treating the patient's CLI. In addition or alternatively, such statistical analysis may also be used to provide real-time thermal image processing to guide stimulation and sweet spot searching to provide objective feedback to guide lead placement and/or SCS programming

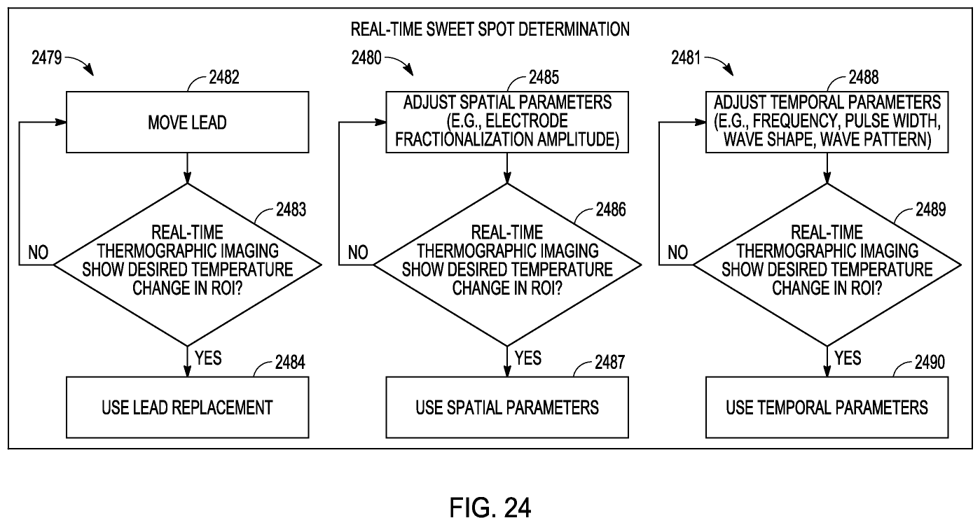

FIG. 24 illustrates, by way of example and not limitation, some real-time sweet spot determinations for lead placement 2479, for programming spatial parameters for the neuromodulation field 2480, and for programming temporal parameters for the neuromodulation field 2481. Neuromodulation energy may be delivered using electrical energy that may have stimulation parameters to specify spatial (where to stimulate), temporal (when to stimulate) and/or informational (stimulation patterns directing the nervous system to respond as desired) aspects of a pattern of neuromodulation pulses.

The real-time sweet spot determination for lead placement 2479 may be performed during an implantation procedure when a surgeon is moving the lead 2482 into a desired position for delivering the SCS. At 2483, a determination is made whether real-time thermographic imaging is showing a desired temperature change in a ROI. The surgeon/clinician may monitor a thermogram during the procedure to confirm that the SCS is implanted in a desirable region. As SCS includes a plurality of electrodes on a lead, the surgeon/clinician may simply be subjectively looking for a significant change in color. Some embodiments may analyze the thermograms using statistical techniques, such as techniques described above. When it is determined that that lead is properly positioned (e.g., at or near a sweet spot), then that lead placement may be used 2484 for the SCS therapy.

The real-time sweet spot determination for programming spatial parameters for the neuromodulation field 2480 may be performed after lead placement for initial programming or may be performed as a follow-up reprogramming. Spatial parameters affect the location of the neuromodulation field, and include the electrodes selected to be active electrodes, the polarity of each active electrode, the fractionalized energy contribution of each active electrode (e.g., an electrode may be programmed to provide X % of the total anodic energy or Y % of the total cathodic energy), and amplitude. During programming, spatial parameters may be adjusted 2485 until the real-time thermographic imaging shows a desired change in the ROI 2486. The surgeon/clinician may subjectively look for a significant change in color. Some embodiments may analyze the thermograms using statistical techniques, such as techniques described above. When it is determined that that the spatial parameters are at or near a sweet spot, then those spatial parameters may be used 2487 for the SCS therapy.

The real-time sweet spot determination for programming temporal parameters for the neuromodulation field 2481 may be performed after lead placement for initial programming or may be performed as a follow-up reprogramming. Programmable temporal parameters may include frequency, pulse with, burst frequency, and ON/OFF duty cycle for intermittent stimulation where an ON cycle may include one or more pulses delivered at a pulse frequency. Temporal parameters may include a variety of neurostimulation patterns, including regular pulse patterns, irregular pulse patterns, and may include a variety of waveform shapes. During programming, spatial parameters may be adjusted 2488 until the real-time thermographic imaging shows a desired change in the ROI 2489. The surgeon/clinician may subjectively look for a significant change in color. Some embodiments may analyze the thermograms using statistical techniques, such as techniques described above. When it is determined that that the spatial parameters are at or near a sweet spot, then those temporal parameters may be used 2490 for the SCS therapy.

Thermographic systems were described above as a specific example for using blood flow imaging to detect or estimate blood flow over a region of the patient. The present subject matter may be implemented with other blood flow imaging (BFI) systems.

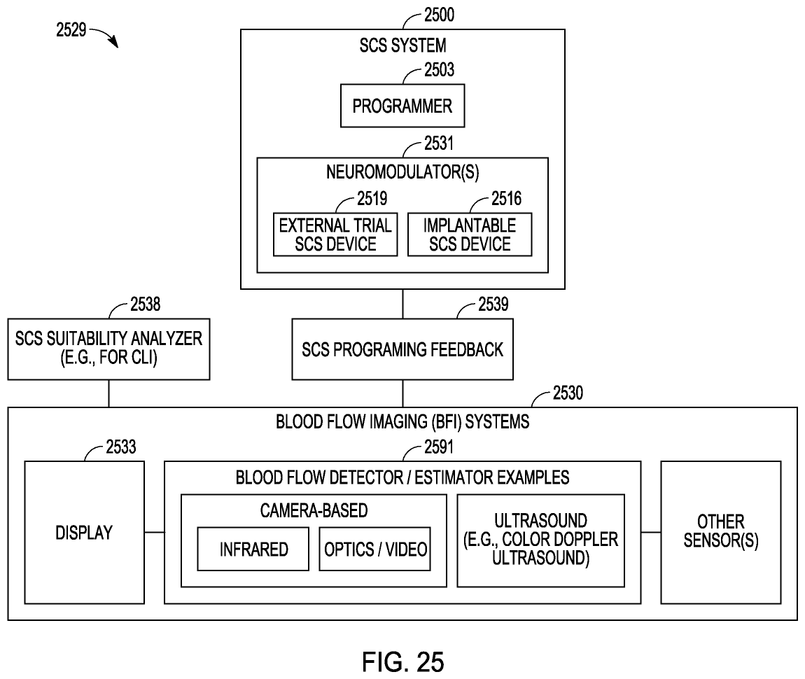

FIG. 25 illustrates a system 2529 that includes both a SCS system 2500 and a blood flow imaging system 2530, similar to the system illustrated in FIG. 6. The illustrated SCS system 2500 may include a programmer 2503 and neuromodulators 2531, including an external trial SCS device 2519 (e.g., ETM 519 in FIG. 5) used to test SCS and an implantable SCS device 2516 (e.g., IPG 516 in FIG. 5). The blood flow imaging system 2530 may generally include a blood flow detector or blood flow estimator 2591 configured to detect or estimate the blood flow in a region of a patient and provide a corresponding image on the display 2533. The blood flow detector/estimator 2591 is capable of acquiring a plurality of pixels of information for the region of the patient, and use the pixels to generate the blood flow image on the display 2533. The illustrated system 2529 may include an SCS suitability analyzer 2538 which may, for example, be used to determine if SCS is suitable for a patient with CLI, and/or may include SCS feedback 2539 (e.g., real-time images) such as may be used for finding sweet spots during programming or implantation procedures. The information may be received from the image on the display 2533 and/or may be received as digital signals from the blood flow detector/estimator 2591. In some embodiments, a user may view the display 2533 and use the image on the display to determine if the patient is suitable for SCS and/or provide feedback for finding sweet spots. In some embodiments the system may include processor(s) configured to analyze information (e.g., digital image information) transmitted in a communication signal. By way of example and not limitation, blood flow detectors/estimators may include camera-based systems such as an infrared camera or an optical camera such as a video camera, or an ultrasound system such as a color doppler ultrasound. Other sensor(s) may be used in conjunction with the detector/estimator to further enhance the accuracy of the blood flow detector/estimator (e.g., see Kumar, M., Suliburk, J. W., Veeraraghavan, A. et al. PulseCam: a camera-based, motion-robust and highly sensitive blood perfusion imaging modality. Sci Rep 10, 4825 (2020). https://doi.org/10.1038/s41598-020-61576-0). Non-contact systems for providing blood flow imaging may record more pixels of information using systems that are economical and accessible. These BFI systems may be used in place of the thermographic imaging systems discussed above.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A system, comprising:
a spinal cord stimulation (SCS) system configured to deliver spinal cord neuromodulation including deliver a test SCS;
a thermographic imaging system configured for taking thermal images of at least a portion of a patient; and
an SCS suitability analyzer configured to:
create thermographic imaging comparison data by comparing a thermographic imaging response to the test SCS to a thermographic imaging baseline before the test SCS is delivered; and
analyze the thermographic imaging comparison data for an indicator of a significant perfusion response to the test SCS to determine whether the patient is a suitable candidate for SCS.

2. The system of claim 1, wherein the thermographic imaging system includes an infrared camera operably connected to a display screen for displaying thermographic imaging, and the thermographic imaging system includes the SCS suitability analyzer.

3. The system of claim 1, wherein the thermographic imaging system includes an infrared camera operably connected to a display screen for displaying thermographic imaging, and the SCS suitability analyzer includes a cloud-based analyzer implemented by one or more remote processing systems, wherein the thermographic imaging system is configured to:
capture and create digital image files for both the thermographic imaging baseline and the thermographic imaging response; and
send the digital image files to the cloud-based analyzer.

4. The system of claim 1, wherein:
the thermographic imaging system includes an infrared camera operably connected to a display screen for displaying thermographic imaging;
the SCS suitability analyzer includes a cloud-based SCS suitability analyzer implemented by one or more remote processing systems;
the SCS system includes a programmer and a neuromodulator configured to deliver SCS including the test SCS; and
the programmer includes a digital camera to capture thermographic imaging displayed on the display screen of the thermographic imaging system, wherein the programmer is configured to use the digital camera create digital image files for both the thermographic imaging baseline and the thermographic imaging response, and configured to send the digital image files to the cloud-based SCS suitability analyzer.

5. The system of claim 1, wherein the thermographic imaging system includes an infrared camera operably connected to a display screen for displaying thermographic imaging, the system further comprising a digital camera to capture thermographic imaging displayed on the display screen of the thermographic imaging system, wherein the digital camera is used to capture and create digital image files for both the thermographic imaging baseline and the thermographic imaging response when displayed on the display screen, and the SCS suitability analyzer is configured to use the digital image files to compare the thermographic imaging response to the test SCS.

6. A method, comprising:
capturing a thermographic imaging baseline, using a thermal imaging system, before delivering a test spinal cord stimulation (SCS);
delivering the test SCS using an SCS system;
capturing a thermographic imaging response to the test SCS using the thermographic imaging system; and
using an SCS suitability analyzer to:
create thermographic imaging comparison data by comparing the thermographic imaging response to the thermographic imaging baseline;
analyze the thermographic imaging comparison data for a perfusion response; and
determine if a patient is a suitable candidate for SCS based on the analyzed thermographic imaging comparison data.

7. The method of claim 6, wherein:
the SCS system includes a programmer and a neuromodulator configured to deliver SCS including the test SCS; and
the programmer of the SCS system includes the SCS suitability analyzer.

8. The method of claim 6, wherein the thermographic imaging system includes an infrared camera operably connected to a display screen for displaying thermographic imaging, and the thermographic imaging system includes the SCS suitability analyzer.

9. The method of claim 6, wherein the thermographic imaging system includes an infrared camera operably connected to a display screen for displaying thermographic imaging, and the SCS suitability analyzer includes a cloud-based analyzer implemented by one or more remote processing systems, wherein the method further comprises using the thermographic imaging system to:
capture and create digital image files for both the thermographic imaging baseline and the thermographic imaging response; and
send the digital image files to the cloud-based analyzer.

10. The method of claim 6, wherein:
the thermographic imaging system includes an infrared camera operably connected to a display screen for displaying thermographic imaging;
the SCS suitability analyzer includes a cloud-based SCS suitability analyzer implemented by one or more remote processing systems;
the SCS system includes a programmer and a neuromodulator configured to deliver SCS including the test SCS; and
the programmer includes a digital camera to capture thermographic imaging displayed on the display screen of the thermographic imaging system, wherein the method further includes using the programmer to create digital image files for both the thermographic imaging baseline and the thermographic imaging response and to send the digital image files to the cloud-based SCS suitability analyzer.

11. The method of claim 6, wherein the thermographic imaging system includes an infrared camera operably connected to a display screen for displaying thermographic imaging, the system further comprising a digital camera to capture thermographic imaging displayed on the display screen of the thermographic imaging system, wherein the method further includes using the digital camera to capture and create digital image files for both the thermographic imaging baseline and the thermographic imaging response when displayed on the display screen, and using the digital image files to compare the thermographic imaging response to the test SCS.

12. The method of claim 11, wherein a personal device includes the digital camera.

13. The method of claim 12, wherein the personal device includes a phone or a tablet, and the personal device includes a downloadable app, wherein the method includes using the downloadable app to create and analyze the thermographic imaging comparison data from the digital image files, or using the downloadable app to send the digital image files to a cloud-based SCS suitability analyzer implemented by one or more remote processing systems.

14. The method of claim 6, wherein the SCS suitability analyzer is used to create and analyze the thermographic imaging comparison data for a determined region of interest (ROI).

15. The method of claim 14, wherein the ROI is automatically determined by automatically determining out-of-norm areas in the thermographic imaging baseline.

16. The method of claim 14, wherein the ROI is determined using user input identifying the determined ROI, and the user input includes:
a user selection of one or more of pre-defined regions; or
a user-defined ROI.

17. The method of claim 6, further comprising programming neuromodulation using real-time thermographic imaging feedback.

18. The method of claim 6, further comprising using the SCS suitability analyzer to:
store pre-SCS thermographic data corresponding the thermographic imaging baseline and SCS-response thermographic data corresponding to the thermographic imaging response;
identify one or more pre-SCS statistical values for the pre-SCS thermographic data and one or more SCS-response statistical value(s) for the SCS-response thermographic data;
determine one or more differences between the pre-SCS thermographic data and the SCS-response thermographic data; and
compare the one or more differences to one or more threshold values to determine if the patient is suitable for SCS.

19. The method of claim 6, further comprising using the SCS suitability analyzer to:
store pre-SCS thermographic data corresponding the thermographic imaging baseline and SCS-response thermographic data corresponding to the thermographic imaging response;
fit the pre-SCS thermographic data to a first distribution curve and fit the SCS-response thermographic data to a second distribution curve;
identify a first set of one or more distribution parameter(s) for the first distribution curve and a second set of the one or more distribution parameters for the second distribution curve;
determine one or more differences between corresponding one or more distribution parameters in the first set and the second set; and
compare the one or more differences to one or more threshold values to determine if the patient is suitable for SCS.

20. The method of claim 19, wherein the first distribution curve and the second distribution curves are gamma distributions, and the one or more distribution parameters include a shape parameter (k) and a scale parameter (Θ).
